(19) United States
(12) Patent Application Publication
(10) Pub. No.: US 2026/0256654 A1
(43) Pub. Date: Sep. 3, 2026

(54) TABLET DISPENSER

(71) Applicant: ALGOCARE CORPORATION, Seoul (KR)

(72) Inventors: Jungjoon HONG, Seongnam-si (KR); Sungmin KANG, Anyang-si (KR)

(73) Assignee: ALGOCARE CORPORATION, Seoul (KR)

(21) Appl. No.: 19/163,692

(22) PCT Filed: Mar. 15, 2024
(86) PCT No.: PCT/KR2024/095571

§ 371 Date: Sep. 9, 2025

(30) Foreign Application Priority Data
Mar. 15, 2023 (KR) 10-2023-0033971

(51) Int. Cl. A61J 7/00; B65D 83/04

(52) U.S. Cl. CPC A61J 7/0084; B65D 83/0409

(57) ABSTRACT

The present invention is directed to a tablet dispenser. The tablet dispenser includes: a discharge part sealing unit positioned beneath a plurality of tablet discharge devices each including a discharge part for discharging stored tablets, and provided with a plurality of openings; and a tablet collection module positioned beneath the discharge part sealing unit. When the discharge part sealing unit is moved in a first planar direction, it blocks the discharge parts to spatially separate the plurality of discharge parts from the tablet collection module. When the discharge part sealing unit is moved in a second planar direction opposite the first planar direction, it positions the openings in the same lines as the corresponding discharge parts to allow the plurality of discharge parts to spatially communicate with the tablet collection module.

### TECHNICAL FIELD

The present invention relates to a tablet dispenser.

### BACKGROUND ART

Nutrients containing trace elements (zinc, iron, magnesium, and/or the like), vitamins, and/or the like are generally recognized as health supplements that supplement nutrients in the human body.

However, these nutrients may be used as treatments when necessary. When a nutrient is used as a treatment, the dosage thereof may be strictly limited depending on a patient's conditions.

For example, patients with osteomalacia must be prescribed vitamin D and take it. The dosage of vitamin D is determined according to the calcium concentration in the blood. In this case, when a patient with osteomalacia takes an excessive amount of vitamin D, side effects such as calcium stones or hypertensive disease may occur.

In addition, isoniazid, which is used to treat tuberculosis, can rapidly consume niacin (nicotinic acid) contained in a patient's blood during the treatment of tuberculosis. When niacin deficiency occurs, a doctor determines the appropriate dosage of isoniazid for the patient based on the concentration of niacin in the patient's blood.

Likewise, there is a risk that a nutritional supplement taken for health may act as a drug that harm a user's health if taken incorrectly depending on the user's physical condition such as underlying disease. It is important to take a required type of nutritional supplement for the human body at a required dosage.

As described above, it is desirable to take tablets, such as health supplements or nutritional supplements or pills taken for treatment purposes, at an appropriate dosage at designated times according to a user's physical conditions.

However, it is difficult to accurately take tablets of a type that is suitable for a user.

In particular, when tablets are taken as health supplements such as nutritional supplements, they do not directly influence the current health of the human body unlike in the case of a disease. Accordingly, there are many cases in which users choose and take products at their own discretion instead of prescriptions from professionals such as pharmacists or doctors. Additionally, it is more difficult to consistently take corresponding tablets in an accurate way.

### DISCLOSURE

### Technical Problem

A technical problem to be solved by the present invention is to simplify the structure of a tablet dispenser that automatically discharges tablets.

### Technical Solution

According to an aspect of the present invention, there is provided a tablet dispenser, including: a discharge part sealing unit positioned beneath a plurality of tablet discharge devices each including a discharge part for discharging stored tablets, and provided with a plurality of openings; and a tablet collection module positioned beneath the discharge part sealing unit; wherein, when the discharge part sealing unit is moved in a first planar direction, it blocks the discharge parts to spatially separate the plurality of discharge parts from the tablet collection module; and wherein, when the discharge part sealing unit is moved in a second planar direction opposite the first planar direction, it positions the openings in the same lines as the corresponding discharge parts to allow the plurality of discharge parts to spatially communicate with the tablet collection module.

The first and second planar directions may be rotation directions in which the discharge part sealing unit is rotated in respective predetermined directions.

Rotation angles in the first and second planar directions may be 10 to 15 degrees.

The tablet collection module may be magnetically coupled to the discharge part sealing unit.

The tablet collection module may include a tablet storage portion that is positioned beneath the outer portion of the discharge part sealing unit and has a first discharge hole and an inclined bottom surface.

The tablet collection module may further include a blocking portion that is coupled to the tablet storage portion and includes a second discharge hole positioned at a location corresponding to the first discharge hole.

The blocking portion may further include at least one of a magnet mounting portion, in which a magnet is positioned, and a handle portion.

The tablet dispenser may further include a printed circuit board mounting part positioned above the discharge part sealing unit and configured such that a printed circuit board is mounted thereon, and the discharge part sealing unit may be coupled to the printed circuit board mounting part and control the opening and closing of the plurality of discharge parts depending on the movement in a first planar direction or a second planar direction.

The printed circuit board mounting part may include a plurality of sealing part mounting holes, and the discharge part sealing unit further may include a plurality of coupling protrusions that are inserted and moved into or in the respective sealing part mounting holes.

Each of the discharge parts may include a discharge tube having a tubular shape, and the printed circuit board mounting part may further include a plurality of insertion holes into each of which the discharge tube is inserted and which maintain a spatial communication state or a separate state with the openings depending on a movement of the discharge part sealing unit.

The tablet dispenser may further include a plurality of sealing parts configured to be inserted into the respective insertion holes of the printed circuit board mounting part.

The discharge part sealing unit may further include first and second movement state detection units which are each positioned above the discharge part sealing unit and whose operating states change depending on a movement state in the first planar direction and a movement state in the second planar direction.

The first and second movement state detection units may be limit switches that each maintain an ON or OFF state depending on the movement state in the first planar direction and the movement state in the second planar direction.

### Advantageous Effects

According to these features, when the tablet discharge device is in a non-operation state, the discharge part connected to the outside is spatially blocked by sealing the discharge part. Accordingly, this prevents foreign substances such as moisture or dust from entering the tablet discharge device via the discharge part.

This significantly reduces the risk in which the tablets positioned inside the tablet discharge device are degraded by moisture or a foreign substance, thereby extending the shelf life of the tablets.

Furthermore, the tablets of the tablet discharge device are protected from not only foreign substances but also pests and insects such as ants, thus ensuring the hygienic storage of the tablets inside the tablet discharge device.

### DESCRIPTION OF DRAWINGS

FIG. 1 is a perspective view of a tablet dispenser according to one embodiment of the present invention, which is a view showing a state in which a portion of a tablet discharge device mounted inside the tablet dispenser is detached with the cover of the tablet dispenser being open;

FIGS. 2a and 2b are perspective views showing a state in which some components are removed from the tablet dispenser shown in FIG. 1;

FIG. 3 is a sectional view obtained when the tablet discharge device shown in FIG. 1 is cut in one direction;

FIG. 4 is a conceptual diagram illustrating the features of the tablet dispenser of the present invention, wherein FIG. 4(a) is a diagram showing a state in which the discharge parts of tablet discharge devices are closed by a movable body and FIG. 4(b) is a diagram showing a state in which the discharge parts of the tablet discharge devices are opened by the movable body;

FIG. 5 is an exploded perspective view of a PCB module, a discharge part sealing unit, and a tablet collection module in the tablet dispenser of FIG. 1;

FIG. 6 is an exploded perspective view of the PCB module, the discharge part sealing unit, and the tablet collection module in the tablet dispenser of FIG. 1, which is a bottom perspective view;

FIG. 7 is a perspective view of a state in which the tablet discharge device is mounted on the PCB module, the discharge part sealing unit, and the tablet collection module, which are coupled to each other, in the tablet dispenser of FIG. 1;

FIG. 8 is a sectional view taken along one direction of FIG. 7, which is a sectional view showing a state in which the discharge part of a tablet discharge device is closed by the discharge part sealing unit;

FIG. 9 is a sectional view taken along one direction of FIG. 7, which is a sectional view showing a state in which the discharge part of the tablet discharge device is opened by the discharge part sealing unit; and

FIG. 10 is a bottom view of a case where a tablet discharge device is coupled to a PCB module in the tablet dispenser of the present invention, wherein FIG. 10(a) is a diagram of a state in which the discharge part of a tablet discharge device is closed by a movable body and FIG. 10(b) is a diagram of a state in which the discharge part of the tablet discharge device is opened by the movable body.

### BEST MODE

The embodiments disclosed herein will be described in detail below with reference to the accompanying drawings. Regardless of the numbers of the drawings, identical or similar components will be given the same reference numerals and redundant descriptions thereof will omitted. Additionally, in the description of the embodiments disclosed herein, when it is determined that a detailed description of a related known technology may obscure the gist of the embodiments disclosed herein, the detailed description will be omitted.

The terms each including an ordinal number, such as first, second, and/or the like, may be used to describe various components, but the components are not limited by the terms. The above terms are each used only for the purpose of distinguishing one component from another.

A singular expression includes a plural expression unless the context clearly dictates otherwise.

In the present application, the individual steps described may be performed regardless of a listed order, except for a case where individual steps need to be performed in a listed order due to a special causal relationship.

It should be understood that in the present application, the terms such as “include,” “have,” or the like are intended to designate the presence of features, numbers, steps, operations, components, parts, or combinations thereof described herein, but are not intended to exclude in advance the possibility of the presence or addition of one or more elements, numbers, steps, operations, components, parts, or combinations thereof.

A tablet dispenser according to one embodiment of the present invention will be described below with reference to the accompanying drawings.

In the present specification, a tablet refers to a health supplement, such as a nutritional supplement, or a medicine made in a solid form according to the purpose of use or purpose thereof. The shapes of such tablets may include various shapes such as circular, oval, cylindrical, and polygonal shapes, etc.

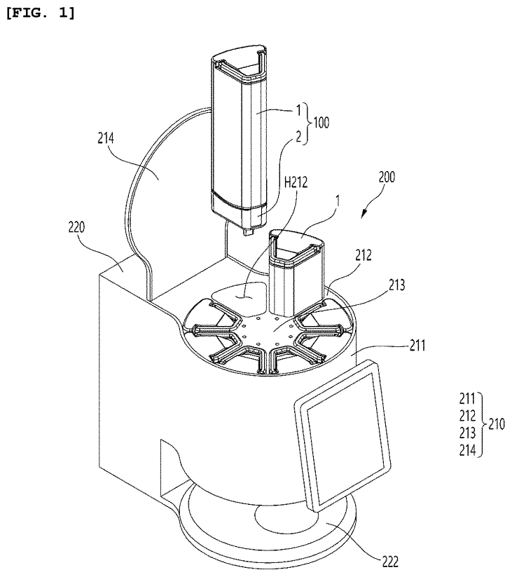

Referring to FIG. 1, a tablet dispenser according to one embodiment of the present invention will be described.

An example of a tablet dispenser 200 may include a storage unit 210 and a main body 220, as shown in FIG. 1.

The storage unit 210 may be a portion into which at least one tablet discharge device 100, each containing tables, is inserted and stored.

The main body 220 is intended to securely maintain the installation state of the tablet discharge device 100 mounted into the storage unit 210 and control the operation of the tablet dispenser 200.

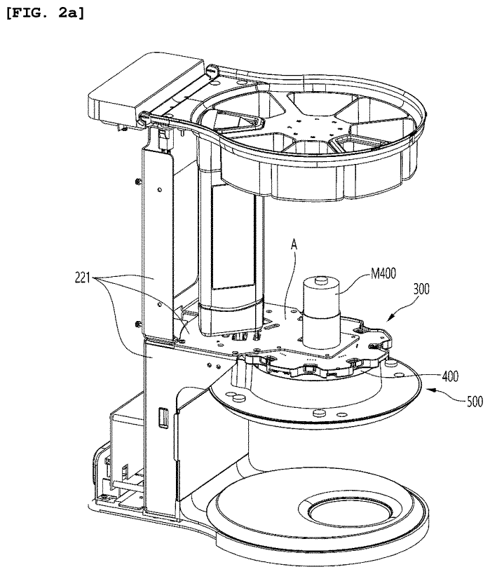

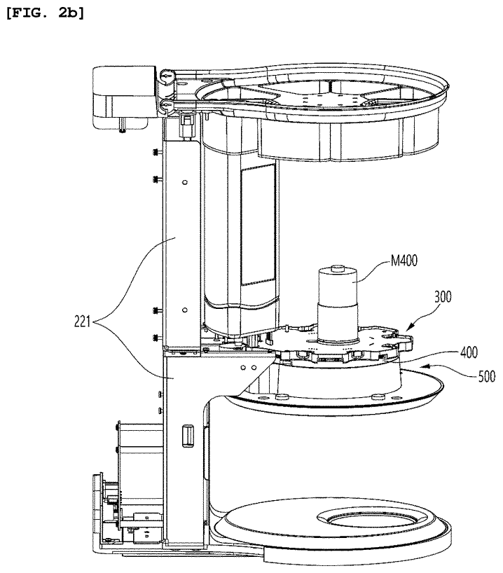

Accordingly, the main body 220 may include a plurality of mounting frames 221, installed in respective predetermined directions, as shown in FIGS. 2a and 2b.

This allows the tablet discharge device 100, mounted within the tablet dispenser 200, to be positioned and stably maintained on the corresponding mounting frame 221.

Furthermore, a support 222 is mounted onto the mounting frames 221, and allows the tablet dispenser 200 to be stably positioned at a desired location.

The storage unit 210 may include a housing 211, a storage portion 212 positioned within the internal space enclosed by the housing 211, a plurality of mounting state indicators 213 positioned in the center of the storage portion 212 and each configured to indicate the mounting state of the tablet discharge device 100 within the storage portion 212, and an openable cover 214 connected to the main body 220.

The housing 211 may form the outer case of the tablet dispenser 200, and may protect various structures positioned within it.

The storage unit 212 is a portion into which the tablet discharge device 100 is inserted and mounted, and may include a plurality of discharge device storage openings H212.

Accordingly, one tablet discharge device 100 may be inserted and stored in each of the discharge device storage openings H212.

In the present example, the discharge device storage openings H212 may be arranged radially around the center of the storage unit 212, but are not limited to this arrangement and may be arranged in various arrangements.

Accordingly, a user may open the cover 214, may insert the tablet discharge device 100 containing a desired type of tablets into the discharge device storage hole H212 at a desired location, and may then close the cover 214 to allow the tablet dispenser 200 to be used.

Furthermore, when the tablets contained in the tablet discharge device 100 are completely exhausted, the user may open the cover 214 again, may remove the tablet discharge device 100 from the discharge device storage opening H212, and may refill the tablet discharge device 100 with the corresponding type of tablets and then mount the tablet discharge device 100 into the corresponding discharge device storage opening H212, or may mount a new tablet discharge device 100 containing the corresponding type of tablets into the corresponding discharge device storage opening H212, thereby enabling the use of the tablet dispenser 200.

The plurality of mounting state indicators 213 may indicate whether tablet discharge devices 100 are mounted in the respective discharge device storage openings H212.

These mounting state indicators 213 may each include at least an indicator element such as a light emitting diode (LED), and may each change its ON/OFF state or emit light of different colors depending on whether the tablet discharge device 100 has been mounted in the corresponding discharge device storage opening H212.

For example, when the tablet discharge device 100 is mounted in the discharge device storage opening H212, the mounting state indicator 213 may remain an OFF state or emit light of a first color (e.g., green). In contrast, when the tablet discharge device 100 is not mounted in the discharge device storage opening H212, the mounting state indicator 213 may remain an ON state or emit light of a first color (e.g., red).

As already described, the tablet discharge device 100 contains a desired type of tablets and is inserted into the discharge device storage opening H212 of the tablet dispenser 200, thereby allowing the contained tablets to be discharged one by one and to be discharged out of the tablet dispenser 200.

This tablet discharge device 100 may be a consumable. In this case, once all the tablets contained in the tablet discharge device 100 have been discharged, it may be replaced with a new product and be inserted into and used in the corresponding discharge device storage opening H212.

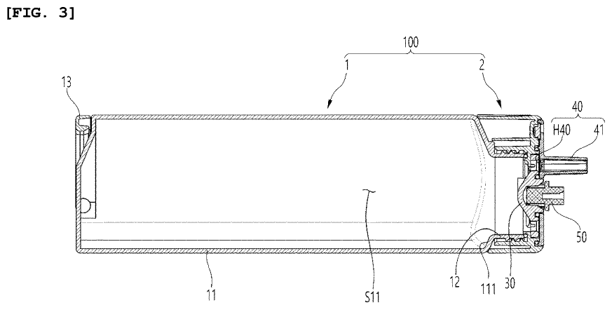

The tablet discharge device 100 of the present example may include a tablet cartridge 1 and a discharge connector 2 connected to the tablet cartridge 1, as shown in FIG. 3.

The discharge connector 2 is coupled to the tablet cartridge 1, and may be detachable as needed. Accordingly, one side (e.g., the upper side) of the discharge connector 2 may be coupled to the tablet cartridge 1, and the other side (e.g., the lower side) of the discharge connector 2 may be positioned in contact with or adjacent to the housing 211.

The tablet cartridge 1 and the discharge connector 2 may be manufactured as separate components.

As an example, the tablet cartridge 1 containing a corresponding type of tablets may be provided in the state of being sealed with a stopper (e.g., a bottle top) other than the discharge connector 2. Accordingly, the user may remove the stopper that blocks the opening of the tablet cartridge 1, may couple the discharge connector 2 to the tablet cartridge 1, may complete the tablet discharge device 100, and may then insert and use the tablet discharge device 100 into and in the appropriate location of the tablet dispenser 200.

At least one of the tablet t cartridge 1 and the discharge connector 2 may be consumable. Once the operation of discharging the tablets contained in the tablet cartridge 1 has been completed, the user may complete the tablet discharge device 100 by using a new product.

Accordingly, as an example, when the tablets contained in the first one 1 of a plurality of tablet cartridges 1 are completely exhausted, the first cartridge 1 and the discharge connector 2, e.g., a first discharge connector 2, connected to the first cartridge 1 may also be discarded.

Therefore, the user may obtain a second cartridge 1 and a second discharge connector 2, which are a new cartridge and discharge connector that are different from the first cartridge 1 and the first discharge connector 2.

In this case, the second discharge connector 2 may be equipped with a cartridge identification unit (not shown) that stores cartridge information for the second cartridge to which it is coupled.

Accordingly, the user may assemble a tablet discharge device 100, i.e., a new cartridge assembly, by coupling the new second discharge connector, equipped with a cartridge identification unit storing cartridge information for the second cartridge 1, to the second cartridge 1, i.e., a new cartridge, and may then mount and use the tablet discharge device 100 into and in the tablet dispenser 200.

The tablet cartridge 1 is a part in which the tablets to be discharged to the outside via the discharge connector 2 is positioned, and may include a cartridge body 11, a coupling portion 12 positioned at the end of the cartridge body 11, and a handle portion 13, as shown in FIG. 3.

The cartridge body 11 is a portion in which the tablets to be discharged to the outside are stored, and may be provided with a storage space S11 containing tablets therein because the bottom thereof is open and the top and side portions thereof are enclosed by a top surface and side surfaces.

The coupling portion 12 positioned at the end of the cartridge body 11 may be a portion that has screw grooves and screw threads positioned on the outer side thereof and is intended for screw coupling with the discharge connector 2.

The handle portion 13 may be rotatably attached to the top of the cartridge body 11, and may additionally be detachably attached thereto.

Accordingly, the user may insert or remove the tablet cartridge 1 into or from the discharge device storage opening H212 of the tablet dispenser 200 by using the handle portion 13.

The discharge connector 2 may include a unit body 20 coupled with the coupling portion 12 of the tablet cartridge 1, a rotor part 30 positioned in the bottom of the unit body 20, and a discharge part 40 positioned below the rotor portion 30.

The unit body 20 is a part that is coupled to the tablet cartridge 1, and may be screw-coupled with the coupling portion 12.

Accordingly, the tables contained in the tablet cartridge 1 may move toward the unit body 20 and be positioned on the rotor part 30.

The rotor part 30 is coupled to a rotating shaft part 50 connected to a driving unit such as a motor (not shown), and may be rotated in a corresponding direction by the rotation of the rotating shaft part 50, for example, by a rotational operation.

As the rotor part 30 is rotated, the tablets moved from the tablet cartridge 1 may move toward the discharge part 40 one by one via a hole formed in the rotor part 40.

Accordingly, the tablets moved toward the discharge part 40 may be discharged to the outside via a discharge hole H40 formed in the discharge part 40.

In the present example, a discharge tube 41 is positioned in the discharge hole H40 of the discharge part 40, and thus the tablet passing through the discharge hole H40 may be discharged to the outside along the long, tubular discharge tube 41.

However, in an alternative embodiment, the discharge tube 41 of the discharge part 40 may be omitted.

The tablet dispenser 200 according to the present example is intended to protect the tablets contained in the tablet discharge device 100 from foreign substances such as moisture or dust.

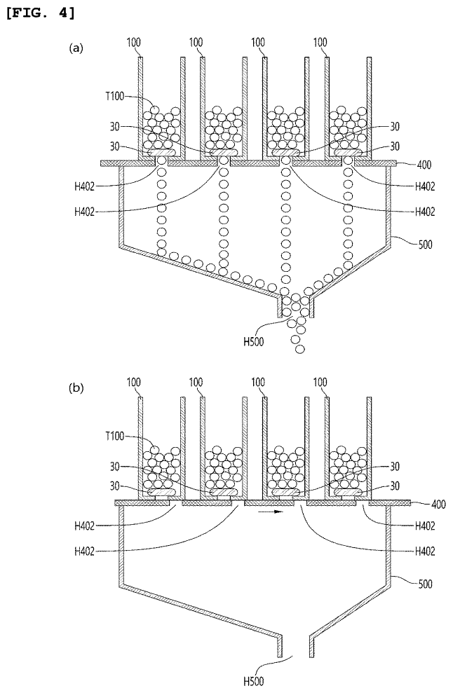

Next, referring to FIG. 4, the concept of the present invention will first be described.

As shown in FIG. 4, the tablet dispenser 200 of the present example may include a movable body 400 positioned beneath at least one tablet discharge device 100, and a tablet collection module 500 positioned beneath the movable body 400.

As already described, each tablet discharge device 100 is provided with the rotor part 30 therein, so that contained tablets T100 can be discharged toward the movable body 400 one by one according to the rotational movement of the rotor part 30.

In the present example, the movable body 400 may be a movable body capable of moving along the planar direction (e.g., moving linearly or rotationally) by the force applied from the outside.

This movable body 400 may be equipped with an opening H402 positioned at a corresponding location facing the discharge part 40 of each tablet discharge device 100. In this case, the top surface of the movable body 400 may be positioned in direct contact with the end of the discharge part 40 of the tablet discharge device 100 or in indirect contact with the end of the discharge part 40 via another component (not shown).

Accordingly, depending on the movement state of the movable body 400, the discharge part 40 of each tablet discharge device 100 may be blocked by an adjacent portion of the movable body 400 or be in communication with the tablet collection module 500 via the facing opening H402.

In this manner, depending on the movement state of the movable body 400, the discharge parts 40 of all tablet discharge devices 100 mounted into the tablet dispenser 200 are simultaneously opened or closed, so that all the discharge parts 40 of the respective tablet discharge devices 100 are either spatially connected to the tablet collection module 500 or spatially separated (i.e., blocked) from the tablet collection module 500.

For example, when the tablet dispenser 200 is in a state in which the tablets T100 are not being discharged, for example, in a non-operation state in which power is not supplied or in a standby state in which power is turned on and the device is waiting for operation, the movable body 400 may maintain its initial state.

In this initial state, the top surface of the movable body 400 may be in the state of blocking the discharge part 40 of the corresponding tablet discharge device 100 positioned on the movable body 400, as shown in FIG. 4a. In this case, the openings H402 positioned in the movable body 400 may be blocked by another component positioned on the movable body 400.

However, when the tablet dispenser 200 is in an operation state for the operation of discharging the tablets T100, the tablet dispenser 200 may move the movable body 400 in a predetermined direction by a predetermined distance, as shown in FIG. 4b. In this case, the movable body 400 may move in a linear direction (e.g., in the up-down or left-right direction) or a rotational direction (in the clockwise or counterclockwise direction) along the planar direction.

Accordingly, by the movement of the movable body 400, each opening H402 of the movable body 400 is positioned in parallel to the discharge part 40 of the corresponding tablet discharge device 100 in the vertical direction, so that one corresponding discharge part 40 and one corresponding opening H402 can maintain a communication state therebetween. That is, when the tablet dispenser 200 is in an operation state, the discharge parts 40 of all the tablet discharge devices 100 that have been blocked by the top surface of the movable body 400 may be opened.

Then, the tablet discharge device 100 is operated, so that the tablets T100 are discharged toward the tablet collection module 500 via the connected discharge parts 40 and the insertion holes H402 in communication with each other and then discharged to the outside via a discharge hole H500 formed in the tablet collection module 500.

In this case, the bottom surface of the tablet collection module 500 may be inclined toward the discharge hole H500, so that the tablets T100 moved to the tablet collection module 500 can easily move toward the discharge hole H500.

Again, when the tablet dispenser 200 is in a non-operation state, the movable body 400 is moved in the direction opposite the direction when it was in an operation state, and returns to its initial location, thus blocking the discharge holes 40 of all the tablet discharge devices.

When the tablets T100 are not discharged from the tablet discharge devices 100 as described above, the discharge parts 40 of all the tablet discharge devices 100 are closed by the movable body 400, so that the tablets T100 positioned inside the tablet discharge devices 100 can be securely stored.

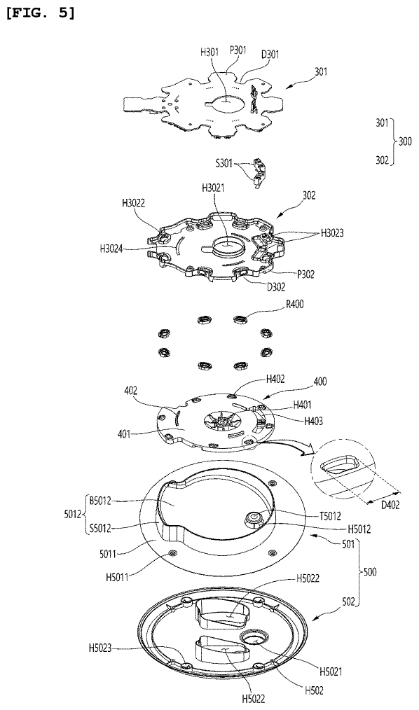

A specific example of the tablet dispenser 200 will be described in detail with additional reference to FIGS. 5 to 10.

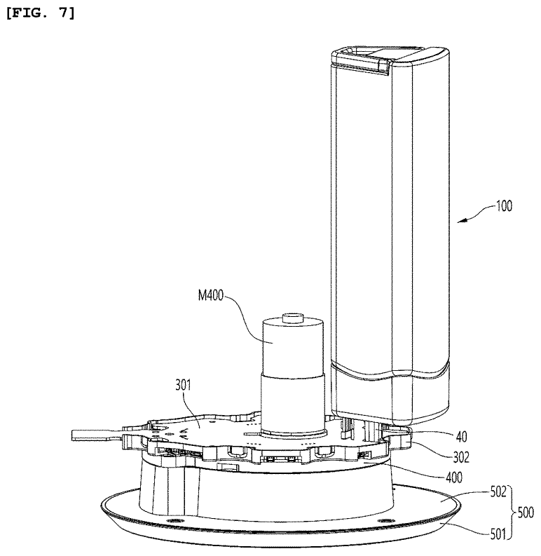

Referring to FIGS. 2a, 2b, and 7, the tablet dispenser 200 of the present example may include a printed circuit board (PCB) module 300 coupled to the mounting frame 221 of the main body 220, a discharge part sealing unit 400 positioned beneath the PCB module 300 and coupled to the PCB module 300, and a tablet collection module 500 positioned beneath the discharge part sealing unit 400.

In the present example, the PCB module 300, the discharge part sealing unit 400, and the tablet collection module 500 may constitute a discharge part sealing device.

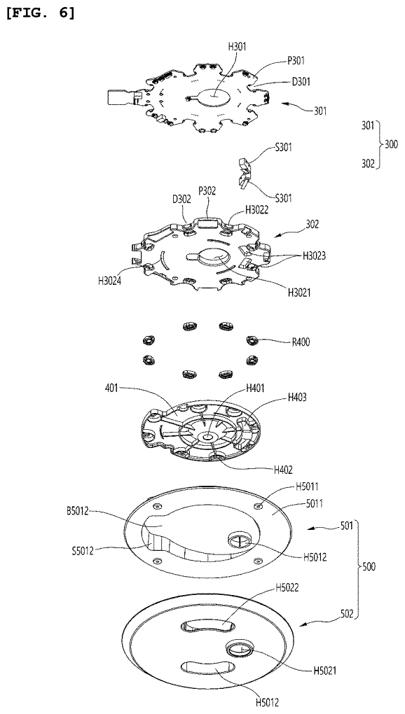

Referring to FIGS. 5 and 6, the PCB module 300 may include a printed circuit board (PCB) 301, and a PCB mounting part 302 configured such that the PCB 301 is mounted thereon.

The PCB 301 may be a rigid PCB, and may be mounted or equipped with various components, such as electric and electronic elements necessary for operating the tablet dispenser 200, a driving chip, connectors for electrical connection with respective tablet discharge devices 100, and signal lines.

Among the components attached to the PCB 301, there may be present a movement state detection unit S301 configured to detect the amount of movement (e.g., the amount of rotation) of the discharge part sealing unit 400 and control the movement of the discharge part sealing unit 400.

When the discharge part sealing unit 400 moves along the planar direction (e.g., moves rotationally or linearly), the movement state detection unit S301 changes its operation state according to the amount of movement and outputs a signal indicating a corresponding state. For example, the movement state detection unit S301 may be a limit switch. Accordingly, a sealing part driving unit M400 may control the operation of the discharge part sealing unit 400 by using a signal applied from the movement state detection unit S301.

In the present example, the movement state detection unit S301 may include two movement state detection units positioned in parallel with each other.

Accordingly, one movement state detection unit S301 is intended to detect the amount of movement (e.g., the amount of rotation) in the first direction (e.g., in the clockwise direction), and the remaining movement state detection unit S301 is intended to detect the amount of movement in the second direction (e.g., in the counterclockwise direction), which is opposite the first direction.

Accordingly, when the corresponding movement state detection unit S301 moves in the corresponding direction by a predetermined amount (e.g., a predetermined angle), the state of the movement state detection unit S30) may change from an initial state (e.g., an OFF state) to an operation state (e.g., an ON state).

This PCB 301 may be positioned in the center, and may include a driving unit mounting hole (e.g., a first driving unit mounting hole H301) into which the sealing part driving unit M400 is mounted.

The sealing part driving unit M400 is intended to move the discharge part sealing unit 400 along the planar direction, and may include a motor.

In the present example, the discharge part sealing unit 400 performs a rotational operation (a forward rotation operation) (e.g., a clockwise rotation operation) or a reverse rotation operation (e.g., a counterclockwise rotation operation) at the angle and in the direction determined by the sealing part driving unit M400, thereby controlling the opening and closing state of the discharge part 40 of the tablet discharge device 100.

However, in an alternative example, the discharge part sealing unit 400 may move in a linear direction (e.g., in the left-right direction or in the up-down direction) along the planar direction to control the opening and closing of the discharge part 400 of the tablet discharge device 100.

Accordingly, the discharge part sealing unit 400 may be a movable body, specifically a rotating body, whose location changes according to the force applied from the sealing part driving unit M400.

Referring back to FIGS. 5 and 6, in the PCB 301, sets of protruding portions P301 protruding outward and a recessed portion D301 positioned between the two protruding portions P301 may be arranged alternately.

As shown in FIGS. 7 to 9, each tablet discharge device 100 may be mounted on a PCB mounting part 302 positioned below after passing through the corresponding recessed portion D301.

Furthermore, a photo sensor including a light-emitting element (e.g., a light-emitting diode) (not shown) and a light-receiving element (e.g., a phototransistor) (not shown) may be mounted on both opposing sides of the recessed portion D301, thereby counting the number of tablets discharged from the discharge part 40 of the tablet discharge device 100.

As already described, the PCB 301 may be mounted on the PCB mounting part 302.

Furthermore, the PCB mounting part 302 may determine whether to open or close the opening H402 positioned in the discharge part sealing unit 400 based on the movement state (e.g., the rotation state) of the discharge part sealing unit 400 under the control of the sealing part driving unit M400.

As shown in FIGS. 5 and 6, the PCB mounting part 302 of the present example may include a driving unit mounting hole (e.g., a second driving unit mounting hole) H3021 in the center into which the sealing part driving unit M400 is inserted.

This second driving unit mounting hole H3021 may be positioned to face the first driving unit mounting hole H301 of the PCB 301 positioned above, and may spatially communicate with the first driving unit mounting hole H301.

As already described, the PCB mounting part 302 is a part on which the PCB 301 is mounted. Accordingly, in the edge of the PCB mounting part 302, sets of a plurality of protruding portions P302 and a recessed portion D302 may be arranged alternately, as in the PCB 301. In this case, each protruding portion P302 and each recessed portion D302 may be positioned to face the corresponding protruding portion P301 and recessed portion D302 of the PCB 301 positioned above.

Furthermore, the PCB mounting part 302 may include a plurality of insertion holes H3022 positioned in portions of the PCB mounting part 302 adjacent to the respective recessed portions D302, movement state detection unit mounting holes H3023, and a plurality of sealing part mounting holes H3024 positioned around the second driving unit mounting hole H3021.

Each of the insertion holes H3022 is a portion into or with which the discharge part 40 of the tablet discharge device 100 is inserted or comes into contact, and may be exposed to the outside via the recessed portion D301 of the PCB 301.

Accordingly, the end of each tablet discharge device 100 may be positioned to come into contact with or be inserted into the corresponding insertion hole H3022 exposed to the outside after passing through the recessed portion D301 of the PCB 301.

The movement state detection unit mounting holes H3023 may be portions into and in which the movement state detection units S301, mounted beneath the PCB 301, are inserted and positioned.

In the present example, the two movement state detection units S301 are positioned separately, and thus the number of movement state detection unit mounting holes H3023 may also be two.

The plurality of sealing part mounting holes H3024 are holes each having a roughly arcuate shape, and may be holes intended to be coupled with the discharge part sealing unit 400 positioned below.

As already described, the discharge part sealing unit 400, which is a movable body, may be coupled to the lower portion of the PCB module 300, i.e., the PCB mounting part 302, and may be moved in a predetermined direction by a predetermined amount according to the operation of the sealing part driving unit M400.

This discharge part sealing unit 400 may include a base substrate 401, and a plurality of coupling protrusions 402 protruding upward from the top surface of the base substrate 401.

The base substrate 401 may have a flat top surface and a shape in which the central portion thereof is sunken downward. A wear prevention portion configured to prevent the wear of the base substrate 401 due to the rotational movement of the sealing part driving unit M400 may be further positioned on the portion of the base substrate 40 having a sunken shape.

In the present example, the discharge part sealing unit 400 may be made of a synthetic resin such as plastic. In this case, when the driving shaft of the sealing part driving unit M400 is inserted into the second driving unit mounting hole H3021 and then rotated, the corresponding portion of the discharge part sealing unit 400, for example, the portion in contact with the second driving unit mounting hole H3021, may wear out, and problems such as the generation of heat due to friction may occur.

The wear protection portion may contain a metal component, such as stainless steel. Accordingly, after the wear protection portion has been mounted on the corresponding portion, the driving shaft of the sealing part driving unit M400 is inserted into the hole positioned in the wear protection portion to enable a rotational operation. In this case, the wear of the discharge part sealing unit 400 due to the rotational operation of the sealing part driving unit M400 is significantly reduced, and the rotational operation of the sealing part driving unit M400 may also be stably performed.

This wear protection portion may have a plate form having a flat shape, such as a circular shape.

The base substrate 401 may include a driving unit mounting portion H401 positioned in the center thereof, openings H402 positioned at the edge thereof, and a second movement state detection unit mounting hole H403 positioned at the edge thereof.

The driving unit mounting part H401 is a part into which the driving shaft of the sealing part driving unit M400 positioned at the top is inserted. It may be a hole that completely passes through the base substrate 401 or a blind hole that only partially passes through it.

Accordingly, the sealing part driving unit M400 may be physically connected to the base substrate 401 of the discharge part sealing unit 400 via the driving unit mounting part H401, and may transmit generated force (e.g., rotational force) to the discharge part sealing unit 400. The discharge part sealing unit 400 may be moved in a predetermined direction (in a forward or reverse rotation direction) by the rotational force transmitted from the sealing part driving unit M400.

Each opening H402 may be positioned at a location corresponding to each insertion hole H3022 positioned above. Each opening H402 may maintain a communication state with the corresponding insertion hole H3022 when the tablet dispenser 200 is in an operation state.

Accordingly, when the tablet dispenser 200 is in an operation state, the end of the discharge part 40 of the tablet discharge device 100 may maintain a spatial communication state with the opening H402.

In the present example, sealing parts R400 may be present between the insertion holes H3022 and the openings H402.

The sealing parts R400 may be made of a material having elasticity like plastic, such as silicone, and may have a ring shape with a hole in the center thereof.

These sealing parts R400 may be inserted into the respective insertion holes H3022, and may increase sealing force with the discharge parts 40 inserted into the corresponding insertion holes H3022.

Accordingly, each of the sealing parts R400 may be mounted such that at least a portion thereof is inserted into the corresponding insertion hole H3022, and may be positioned in contact with the base substrate 401 of the discharge part sealing unit 400 enclosing the opening H402 positioned below.

Due to this sealing part R400, the discharge part 40 inserted into the insertion hole H3022 may be inserted into the insertion hole H3022 more stably and tightly.

In this case, the lower end of the sealing part R400 may be positioned to be lower than the lower end of the PCB mounting part 302. In this case, the top surface of the discharge part sealing unit 400 may be rotated while in contact with the lower end of the sealing part R400.

However, in an alternative example, these sealing parts R400 may be omitted.

The second movement state detection unit mounting hole H403 may be positioned to correspond to the first movement state detection unit mounting hole H3023 positioned above. Accordingly, the terminal of the corresponding movement state detection unit S301, which is exposed through the first movement state detection unit mounting hole H3023, may protrude downward via the second movement state detection unit mounting hole H403.

Accordingly, when the discharge part sealing unit 400 is moved (e.g., rotated) in a predetermined direction by a predetermined amount (e.g., a predetermined angle), the terminal of the corresponding movement state detection unit S301, whose location is fixed, may be compressed by the rotating discharge part sealing unit 400. This terminal compression operation causes the operation state of the corresponding movement state detection unit $301 to change to an ON state.

As described above, when the amount of rotation, which is the amount of movement of the discharge part sealing unit 400, is equal to or exceeds a predetermined amount, the corresponding one of the two movement state detection units S301 enters an ON state, and thus the rotational operation of the discharge part sealing unit 400 may be controlled (e.g., stopped).

In this case, one of the two movement state detection units S301 that turns on depending on the movement direction may be determined.

In the present example, the rotation angle of the discharge part sealing unit 400 may range from 10 to 15 degrees. This rotation angle may be determined by the size (e.g., the size in the rotational direction) D402 of the openings H402. The interval between adjacent openings H402 may be determined by the size D402 of the openings H402.

The plurality of mounting protrusions 402 may be positioned to correspond to the sealing part mounting holes H3024 positioned in the PCB mounting part 302 above, and may be inserted into the corresponding sealing part mounting holes H3024 so that the discharge part sealing unit 400 can move along the corresponding sealing part mounting holes H3024.

In this case, as for the sealing part mounting holes H3024 and the mounting protrusions 402 that face each other and are coupled to each other, the extension length of the sealing part mounting holes H3024 may be longer than that of the mounting protrusions 402. As an example, the additional extension length of the corresponding sealing part mounting holes H3024 may be determined by considering the amounts of movement of the discharge part sealing unit 400 in respective directions.

Next, the tablet collection module 500 is intended to receive the tablets discharged via the discharge part sealing unit 400 and finally discharge them to the outside.

This tablet collection module 500 may be detachably coupled to the discharge part sealing unit 400 positioned above. In the present example, the tablet collection module 500 may be attached to and detached from the discharge part sealing unit 400 by using the magnetic force of a magnet.

Accordingly, the user may detach the tablet collection module 500 from the discharge part sealing unit 400 without using a separate tool whenever desired.

As shown in FIGS. 5 and 6, the tablet collection module 500 may include a tablet storage portion 501, and a blocking portion 502 coupled to the tablet storage portion 501.

The tablet storage portion 501 may include an edge portion 5011, and a storage portion 5012 enclosed with the edge portion 5011.

The edge portion 5011 has a circular, flat shape with a predetermined width, may have a flat surface, and may include a coupling hole H5011 for coupling with the blocking portion 502.

The storage portion 5012 may have an open top, may have a fixed height, and may include a side surface S5012 protruding upward from the edge portion 5011 and a bottom surface B5012 connected to the side surface S5012.

In the present example, the open top of the storage portion 5012 is covered by the discharge part sealing unit 400 positioned above. Accordingly, the inside of the storage portion 5012 may be formed as a space enclosed with the side surface S5012, the bottom surface B5012, and the discharge part sealing unit 400. Therefore, the tablets discharged into the storage portion 5012 are prevented from being moved to the outside.

The side surface S5012 may function as a barrier to prevent the tablets discharged into the storage portion 5012 from flowing outward past the storage portion 5012. Accordingly, the tablets discharged into the storage portion 5012 may be securely positioned within the space enclosed with the side surface S5012.

The bottom surface B5012 may have an inclined surface inclined along a predetermined direction, so that the height of the bottom surface B5012 may vary depending on its location. As an example, the height of the bottom surface on one side (e.g., one side bottom surface) B5012 may be substantially equal to the maximum height of the side surface S5012, and the height of the bottom surface on the other side (e.g., the other side bottom surface) B5012, positioned opposite the one side and facing the one side, may be substantially equal to the minimum height of the side surface S5012.

Accordingly, the tablets fallen into the storage portion 5012 may be moved along the inclined surface toward the other side bottom surface B5012.

As shown in FIG. 6, a discharge hole (e.g., a first discharge hole) H5012 may be positioned in the other side bottom surface B5012, so that the tablets moved toward the other side bottom surface B5012 may be discharged to the outside via the first discharge hole H5012.

A blocking plate T5012 may be positioned above the first discharge hole H5012 to be spaced apart from the bottom surface B5012, enclosing the first discharge hole H5012, by a predetermined distance. In this case, the interval between the top surface of the bottom surface B5012 and the lowest bottom surface of the blocking plate T5012 may be larger than the maximum size of the tablets. This allows the tablets to pass through the first discharge hole H5012 stably and rapidly without the interference of the blocking plate T5012.

The blocking plate T5012 is intended to adjust the time at which the tablet is discharged to the outside, and is intended to prevent the tablet, discharged via the discharge hole 40 of the tablet discharge device 100, from falling directly into the first discharge hole H5012 instead of falling to the bottom surface B5012.

That is, when the tablet is discharged in the state in which the discharge part 40 of the tablet discharge device 100 and the first discharge hole H5012 face each other on opposite sides, the tablet discharged from the discharge part 40 may fall directly into the first discharge port H5012 and be discharged to the outside without hitting the bottom surface B5012.

The time for which the tablet is discharged when it falls directly into the first discharge hole H5012 may be significantly shorter than the time for which the tablet is discharged when it falls toward the inclined bottom surface B5012, may be moved along the bottom surface B5012 toward the first discharge hole H5012, and may be discharged via the first discharge hole H5012.

However, when the blocking plate T5012 is positioned above the first hole H5012, as in the present example, the tablet falling directly toward the first hole H5012 will collide with the blocking plate T5012, bounce in a corresponding direction, fall on the corresponding bottom surface B5012, and move toward the first hole H5012. Due to the function of the blocking plate T5012, the time for which the tablet is discharged may be extended.

Accordingly, the time for which the tablet is discharged via the first hole H5012 may be kept substantially constant by this blocking plate T5012.

The blocking portion 502 may be positioned below the tablet storage portion 501, and may be detachably attached to the tablet storage portion 501.

As already described, the tablet storage portion 501 and the blocking portion 502 may be coupled to each other via a fastening member such as a screw, and may be separated from each other by releasing the fastening member when necessary.

Accordingly, the blocking portion 502 may also have coupling portions H502, such as coupling holes or coupling recesses, in portions corresponding to the coupling holes H5011 of the tablet storage portion 501.

Furthermore, the blocking portion 502 may include a discharge hole (e.g., a second discharge hole) H5021, at least one handle portion H5022, and a plurality of magnet mounting portions H5023 in which magnets are positioned.

The second discharge hole H5021 is positioned at a location corresponding to the first discharge hole H5012 of the tablet storage portion 501, and thus, may be positioned on the same virtual vertical line. Accordingly, the tablet passing through the first discharge hole H5012 may again be passed through the second discharge hole H5021 and then discharged to the outside.

Accordingly, the first discharge hole H5012 and the second discharge hole H5021 may constitute the discharge holes of the tablet storage module 500.

The handle portion H5023 is a portion that serves as a handle that a user can hold with his or her hand. Using this handle portion H5023, the user may easily separate the tablet collection module 500, in which the tablet storage portion 501 and the blocking portion 502 are coupled to each other, from the discharge part sealing unit 400.

As already described, the tablet storage portion 501 is a portion in which the tablet discharged from the tablet discharge device 100 falls and is then discharged to the outside. Accordingly, a large amount of foreign material, such as tablet powder that falls f off the tablet during falling, accumulates in the tablet storage portion 501.

Accordingly, the user may detach the tablet collection module 500 from the discharge part sealing unit 400 by using the handle portion H5022, and may then separate the tablet storage portion 501 and the blocking portion 502. The user may then wash the tablet storage portion 501 and the blocking portion 502 by using water or other substances. Accordingly, this allows for easy and frequent removal of foreign substances from the tablet storage module 500.

The plurality of magnet mounting portions H5023 may be portions in which magnets are positioned.

Accordingly, as already described, the tablet collection module 500 is attached to the discharge part sealing unit 400 positioned above by using magnetic force. Accordingly, the user may conveniently and rapidly detach the tablet collection module 500 attached to the discharge part sealing unit 400 without using a separate tool such as a screwdriver.

In an alternative example, the blocking portion 502 may be omitted, in which case the tablet storage portion 501 may further include at least one of a magnet mounting portion H5023, in which a magnet is installed, and a handle portion H5023.

As already described and shown in FIGS. 7 to 9, a discharge part sealing device having this structure may be equipped with the tablet discharge device 100. Furthermore, depending on the movement of the discharge part sealing unit 400, the discharge part 40 of the tablet discharge device 100 may maintain an open state, in which it is connected to the tablet collection module 500, or a closed state, in which it is blocked from the tablet collection module 500.

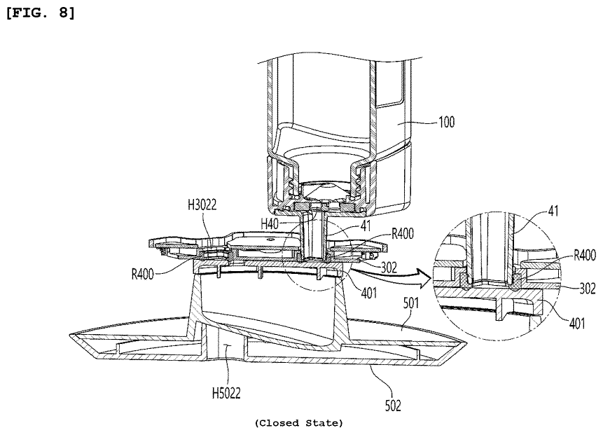

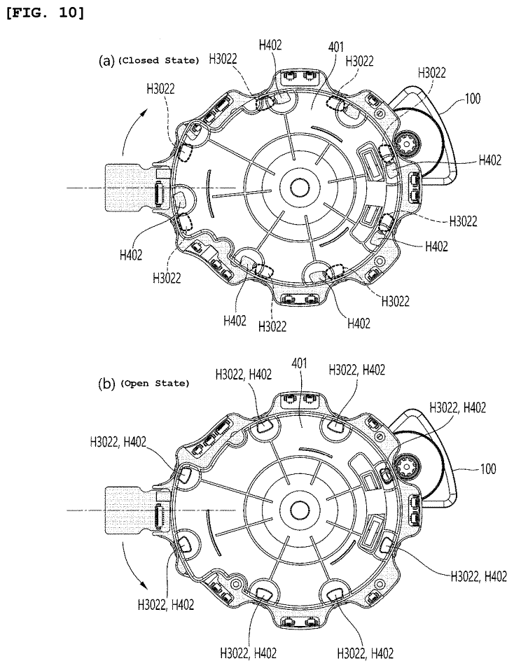

When the tablet dispenser 200 is in a non-operation state, such as a non-driving state or a standby state, the discharge part sealing unit 400 shown in FIGS. 8 and 10(a) may maintain its initial state, thus maintaining a closed state in which all the discharge parts 40 are blocked. In this case, all the openings H402 positioned in the discharge part sealing unit 400 may be blocked by the PCB mounting part 302, which is a component positioned above.

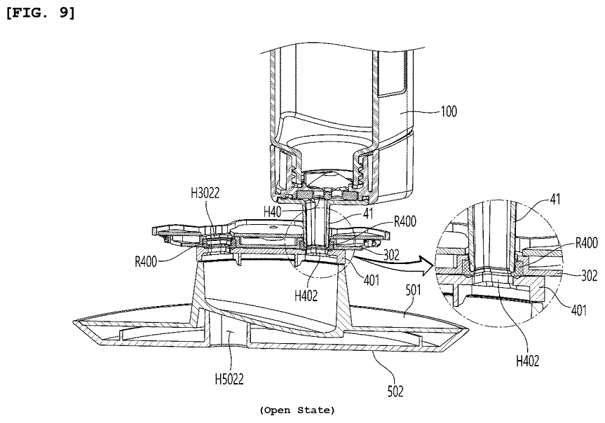

However, as shown in FIGS. 9 and 10(b), when the discharge part sealing unit 400 is moved in a predetermined direction by a predetermined amount, the corresponding discharge parts 40 and the openings H402 of the discharge part sealing unit 400, which are positioned vertically and correspond to each other, may be positioned on the same vertical lines. Accordingly, the discharge parts 40 and openings H402 corresponding to each other may enter a communication state in which they spatially communicate with each other along the vertical directions.

Therefore, the tablet discharged via the discharge part 40 of the tablet discharge device 100 by the operation of the tablet discharge device 100 may be sequentially passed through the corresponding insertion hole H3022 and the corresponding opening H402 and moved downward toward the tablet collection module 500.

The technical features disclosed in the respective embodiments of the present invention are not limited to the corresponding embodiments, and the technical features disclosed in the respective embodiments may be combined and applied to other embodiments unless they are incompatible with each other.

Therefore, while the individual embodiments have been described with a focus on respective technical features, they may be applied in combination with each other unless the technical features are incompatible with each other.

The present invention is not limited to the above-described embodiments and the accompanying drawings, and various modifications and variations may be made from the perspective of a person having ordinary skill in the art to which the present invention pertains. Therefore, the scope of the present invention should be determined not only by the claims of this specification but also by equivalents to these claims.

1. A tablet dispenser, comprising:
a discharge part sealing unit positioned beneath a plurality of tablet discharge devices each including a discharge part for discharging stored tablets, and provided with a plurality of openings; and
a tablet collection module positioned beneath the discharge part sealing unit;
wherein, when the discharge part sealing unit is moved in a first planar direction, it blocks the discharge parts to spatially separate the plurality of discharge parts from the tablet collection module; and
wherein, when the discharge part sealing unit is moved in a second planar direction opposite the first planar direction, it positions the openings in the same lines as the corresponding discharge parts to allow the plurality of discharge parts to spatially communicate with the tablet collection module.

2. The tablet dispenser of claim 1, wherein the first and second planar directions are rotation directions in which the discharge part sealing unit is rotated in predetermined directions.

3. The tablet dispenser of claim 2, wherein rotation angles in the first and second planar directions are 10 to 15 degrees.

4. The tablet dispenser of claim 1, wherein the tablet collection module is magnetically coupled to the discharge part sealing unit.

5. The tablet dispenser of claim 1, wherein the tablet collection module comprises a tablet storage portion that is positioned beneath an outer portion of the discharge part sealing unit and has a first discharge hole and an inclined bottom surface.

6. The tablet dispenser of claim 5, wherein the tablet collection module further comprises a blocking portion that is coupled to the tablet storage portion and includes a second discharge hole positioned at a location corresponding to the first discharge hole.

7. The tablet dispenser of claim 6, wherein the blocking portion further comprises at least one of a magnet mounting portion, in which a magnet is positioned, and a handle portion.

8. The tablet dispenser of claim 1, further comprising a printed circuit board mounting part positioned above the discharge part sealing unit and configured such that a printed circuit board is mounted thereon;
wherein the discharge part sealing unit is coupled to the printed circuit board mounting part and controls opening and closing of the plurality of discharge parts depending on a movement in a first planar direction or a second planar direction.

9. The tablet dispenser of claim 8, wherein:
the printed circuit board mounting part comprises a plurality of sealing part mounting holes; and
the discharge part sealing unit further comprises a plurality of coupling protrusions that are inserted and moved into and in the respective sealing part mounting holes.

10. The tablet dispenser of claim 8, wherein:
each of the discharge parts comprises a discharge tube having a tubular shape; and
the printed circuit board mounting part further comprises a plurality of insertion holes into each of which the discharge tube is inserted and which maintain a spatial communication state or a separate state with the openings depending on a movement of the discharge part sealing unit.

11. The tablet dispenser of claim 10, further comprising a plurality of sealing parts configured to be inserted into the respective insertion holes of the printed circuit board mounting part.

12. The tablet dispenser of claim 2, wherein the discharge part sealing unit further comprises first and second movement state detection units which are each positioned above the discharge part sealing unit and whose operating states change depending on a movement state in the first planar direction and a movement state in the second planar direction.

13. The tablet dispenser of claim 12, wherein the first and second movement state detection units are limit switches that each maintain an ON or OFF state depending on the movement state in the first planar direction and the movement state in the second planar direction.
